(19) United States
(12) Patent Application Publication
(10) Pub. No.: US 2011/0124909 A1
(43) Pub. Date: May 26, 2011

(54) NOVEL PROCESS

(76) Inventors: Vinayak Gore, Maharashtra (IN); Datta Debashish, Maharashtra (IN); Gadakar Maheshkumar, Maharashtra (IN); Pokharkar Kiran, Maharashtra (IN); Mankar Viraj, Maharashtra (IN); Wavhal Sneha, Maharashtra (IN)

(21) Appl. No.: 12/935,764

(22) PCT Filed: Apr. 2, 2009
(86) PCT No.: PCT/GB09/50326

§ 371 Date: Jan. 26, 2011

(30) Foreign Application Priority Data
Apr. 4, 2008 (IN) 679/KOL/2008

(51) Int. Cl. C07C 229/08; C07B 57/00

(52) U.S. Cl. 562/401; 562/553

(57) ABSTRACT

The present invention relates to a novel process of preparing an enantiomerically enriched γ-amino acid, such as enantiomerically enriched (S)-pregabalin.

### CROSS-REFERENCE TO RELATED APPLICATION(S)

This application is a Section 371 National Stage Application of International No. PCT/GB2009/050326, filed 2 Apr. 2009 and published as WO 2009/122215 A1 on 8 Oct. 2009, which claims priority from the IN Patent Application No. 679/KOL/2008, filed 26 Dec. 2007, the contents of which are incorporated herein in their entirety for all purposes.

### FIELD OF THE INVENTION

The present invention relates to a novel process of preparing an enantiomerically enriched γ-amino acid, such as enantiomerically enriched (S)-pregabalin.

### BACKGROUND OF THE INVENTION

Many γ-amino acids, such as (S)-pregabalin (1), are useful as anticonvulsant drugs.

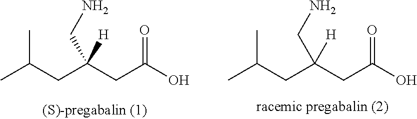

(S)-Pregabalin (1) is related to the endogenous inhibitory neurotransmitter γ-amino-butyric acid (GABA), which is involved in the regulation of brain neuronal activity. (S)-Pregabalin (1) exhibits anti-seizure activity and is also thought to be useful for treating, amongst other conditions, pain, physiological conditions associated with psychomotor stimulants, inflammation, gastrointestinal damage, alcoholism, insomnia, fibromyalgia and various psychiatric disorders, including mania and bipolar disorder.

Various literature articles and patent applications have disclosed synthetic routes to racemic γ-amino acids, such as WO 2000/039074; WO 2004/031124; US 2005/0043565; and Synthesis, 1989, pages 953-955. Processes for the asymmetric synthesis of enantiomerically enriched γ-amino acids, which make use of chiral starting materials, chiral auxiliaries, chiral reagents, or chiral catalysts, or which involve enzyme catalyzed reactions or the resolution of racemic reaction intermediates, have also been reported in various literature articles and patent applications, such as WO 93/23383; WO 2001/055090; WO 2005/087370; WO 2005/100580; WO 2006/110783; WO 2006/121557; WO 2006/122255; WO 2006/122258; WO 2007/035789; WO 2007/035890; WO 2007/139933; WO 2007/143152; U.S. Pat. No. 5,616,793; US 2004/0186177; US 2005/0222464; US 2005/0283023; RU 2146246; Bioorganic & Medical Chemistry Letters, 1994, vol. 4, no. 6, pages 823-826; Bioorganic & Medical Chemistry Letters, 1997, vol. 7, no. 19, pages 2481-2484; J. Med. Chem., 1998, vol. 41, pages 1838-1845; Tetrahedron Asymmetry, 2007, vol. 18, pages 1481-1485; Tetrahedron Letters, 2007, vol. 48, pages 4305-4308; Tetrahedron, 2007, vol. 63, pages 5820-5831; Syn. Lett., 2006, vol. 10, pages 1589-1591; Journal of Molecular Catalysis B: Enzymatic, 2006, vol. 41, pages 75-80; Organic Process Research & Development, 1997, vol. 1, pages 26-38; Journal of American Chemical Society, 2003, vol. 125, pages 10219-10227; Journal of American Chemical Society, 2004, vol. 126, pages 9920-9921; Journal of American Chemical Society, 2007, vol. 129, pages 9216-9221; Journal of Organic Chemistry, 2003, vol. 68, pages 5731-5734; Journal of Organic Chemistry, 2007, vol. 72, pages 7390-7393; and Drugs of the Future, 1999, vol. 24, no. 8, pages 862-870. The resolution of racemic γ-amino acids to obtain enantiomerically enriched γ-amino acids has been suggested only rarely.

U.S. Pat. No. 5,637,767 disclosed a synthesis of racemic pregabalin (2) and the resolution of the racemic pregabalin (2) to obtain (S)-pregabalin (1). The resolution process reported in U.S. Pat. No. 5,637,767 is outlined in Scheme 1. The process involved the use of 1.5 eq. of (S)-mandelic acid in 3% v/v water in isopropanol at 50-65° C. (S)-pregabalin (S)-mandelate (3) was isolated by seeding and cooling the reaction mixture at 20-25° C. for at least 12 hours. The (S)-pregabalin (S)-mandelate (3) isolated after the first resolution step was found to have an (S)-isomer content of around 92.5% by chiral HPLC. Therefore the (S)-pregabalin (S)-mandelate (3) was purified using further (S)-mandelic acid (0.2 eq.) in 3% v/v water in isopropanol at 60-70° C. to obtain enantiomerically purer (S)-pregabalin (S)-mandelate (3) after cooling at 0-5° C. for at least 12 hours. The (S)-pregabalin (S)-mandelate (3) isolated after the second resolution/crystallisation step was found to have an (S)-isomer content of around 98.6% by chiral HPLC. (S)-Pregabalin (1) was isolated by warming the (S)-pregabalin (S)-mandelate (3) in 5% v/v water in tetrahydrofuran at 50-55° C. to obtain (S)-pregabalin (1) after cooling at 0-5° C. for at least 12 hours. For further purification, the (S)-pregabalin (1) was dissolved in 25% v/v water in isopropanol by heating at 75-80° C. and recrystallised by seeding and cooling at 0 to −5° C. The same process of resolving racemic pregabalin (2) using (S)-mandelic acid was also disclosed, for example, in US 2006/0270871; and Organic Process Research & Development, 1997, vol. 1, pages 26-38; and mentioned, for example, in Drugs of the Future, 1999, vol. 24, no. 8, pages 862-870; Journal of Organic Chemistry, 2003, vol. 68, pages 5731-5734; and Tetrahedron Asymmetry, 2007, vol. 18, pages 1481-1485.

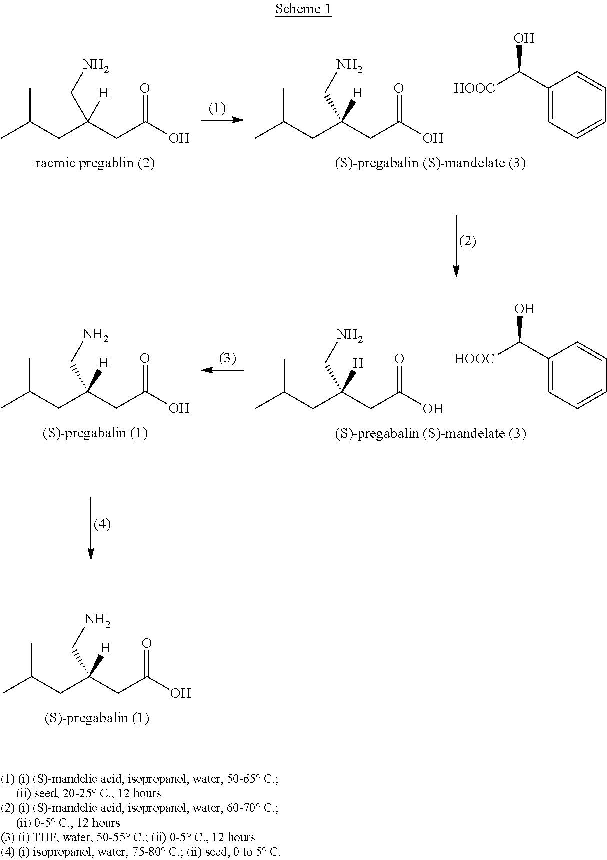

Thus the prior art teaches a huge variety of asymmetric syntheses to prepare enantiomerically enriched γ-amino acids. The resolution of racemic γ-amino acids to obtain enantiomerically enriched γ-amino acids is rarely suggested. However, if the skilled person nevertheless were inclined to make an enantiomerically enriched γ-amino acid by resolving the racemic γ-amino acid, then the prior art teaches the skilled person to use (S)-mandelic acid as resolving agent in 3% v/v water in isopropanol at relatively high temperatures.

However, the process disclosed in U.S. Pat. No. 5,637,767 using (S)-mandelic acid suffers from various disadvantages, which lead to the (S)-pregabalin (1) obtained by the process being too enantiomerically impure and too chemically impure to meet ICH guidelines. Most importantly, the process requires the use of relatively high temperatures (up to 80° C.), which leads to degradation and lactam formation, which makes the process low yielding and renders the (S)-pregabalin (1) obtained impure. The process disclosed in U.S. Pat. No. 5,637,767 also suffers from various disadvantages, which lead to the process being unsuitable for commercial manufacture. As indicated above, the process is low yielding due to degradation and lactam formation due to the use of relatively high temperatures. Moreover, the process takes a long time, with the first resolution step, the second resolution/crystallisation step and the step of isolating (S)-pregabalin (1) requiring at least 12 hours each. Furthermore, the (S)-mandelic acid which is used in excess (1.7 eq.) is an expensive resolving agent.

In view of the importance of γ-amino acids, such as (S)-pregabalin (1), for convulsant treatment, there is a great need for a simple, convenient, inexpensive and commercially viable process for the synthesis of γ-amino acids with a commercially acceptable yield and sufficiently high purity.

### DEFINITIONS

For the purposes of the present invention, a compound with one or more chiral centres is “enantiomerically enriched”, if it comprises more than 75% of one stereoisomer, preferably more than 80%, preferably more than 85%, preferably more than 90%, preferably more than 95%. Accordingly, the term “enantiomerically enriched γ-amino acid” encompasses, for example, “enantiomerically enriched (S)-pregabalin” which comprises more than 75% of the (S)-stereoisomer and less than 25% of the (R)-stereoisomer. Similarly, the term “enantiomerically enriched γ-amino acid salt” encompasses, for example, “enantiomerically enriched (S)-pregabalin (L)-tartrate” which comprises more than 75% of the (S),(L)-stereoisomer and less than 25% of the (S),(D)-, (R),(L)- and (R),(D)-stereoisomers.

For the purposes of the present invention, a compound with one or more chiral centres is “racemic”, if it is not enantiomerically enriched. Preferably a “racemic” compound with one or more chiral centres comprises each stereoisomer relative to each other stereoisomer in a ratio of from 1:1.5 to 1.5:1. This means that a compound with one chiral centre is “racemic”, if it comprises 40-60% of each of the two stereoisomers. A compound with two chiral centres is “racemic”, if it comprises 20-30% of each of the four stereoisomers. Etc. Accordingly, the term “racemic γ-amino acid” encompasses, for example, “racemic pregabalin” which comprises (S)-pregabalin and (R)-pregabalin in a ratio of from 60:40 to 40:60.

For the purposes of the present invention, the term “an enantiomer” of an optically active acid such as tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof, or “an enantiomer” of a mandelic acid derivative, means that the acid or the derivative thereof comprises more than 95% of one stereoisomer, preferably more than 98%, preferably more than 99%, preferably more than 99.9%. Similarly, the term “(L)-tartaric acid” means that the tartaric acid comprises more than 95% of the (L)-stereoisomer, preferably more than 98%, preferably more than 99%, preferably more than 99.9%. Similarly, the term “O,O′-di-p-toluoyl-(D)-tartaric acid” means that the O,O′-di-p-toluoyl-tartaric acid comprises more than 95% of the (D)-stereoisomer, preferably more than 98%, preferably more than 99%, preferably more than 99.9%.

Accordingly the term “pregabalin (L)-tartrate or a derivative thereof, wherein the pregabalin in the pregabalin (L)-tartrate or the derivative thereof is racemic” refers to the salt of racemic pregabalin with (L)-tartaric acid or a derivative thereof, wherein the terms “racemic” and “(L)-tartaric acid” are as defined above.

For the purposes of the present invention, an “acid resolving agent” is any acid capable of forming a salt with the amino group of a γ-amino acid. Preferred acid resolving agents of the present invention are tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, and derivatives thereof, preferably (L)-tartaric acid, O,O′-di-p-toluoyl-(L)-tartaric acid, O,O′-dibenzoyl-(L)-tartaric acid, (S)-3-chloro-mandelic acid, and (S)-3-bromo-mandelic acid, preferably (L)-tartaric acid.

For the purposes of the present invention, a “corresponding lactam” or a “lactam impurity” is the lactam obtained by an intra-molecular condensation reaction of the γ-amino group and the carboxylic acid group of a γ-amino acid.

### SUMMARY OF THE INVENTION

Surprisingly the present inventors have found that enantiomers of optically pure acids other than (S)-mandelic acid are suitable for preparing enantiomerically enriched γ-amino acids, such as enantiomerically enriched (S)-pregabalin (1).

The present invention provides an enantiomerically enriched γ-amino acid, such as (S)-pregabalin (1), by a simple, convenient, scalable and commercially acceptable process.

The present invention also provides a process for the preparation of an enantiomerically enriched γ-amino acid, such as (S)-pregabalin (1), by using a simple resolution technique.

In another aspect, the present invention provides a process for the preparation of an enantiomerically enriched γ-amino acid, such as (S)-pregabalin (1), by using an inexpensive and readily available resolving agent.

In still another aspect, the present invention provides a process for the preparation of an enantiomerically enriched γ-amino acid, such as (S)-pregabalin (1), by using efficient fractional crystallisation of a diastereomeric salt.

In still yet another aspect, the present invention provides a process for the preparation of an enantiomerically enriched γ-amino acid, such as (S)-pregabalin (1), by using a simple, convenient and scalable technique to isolate the enantiomerically enriched γ-amino acid from a diastereomeric salt thereof.

The present invention also provides a process for the preparation of an enantiomerically enriched γ-amino acid, such as (S)-pregabalin (1), which meets the requirements of the ICH guidelines.

The present invention further provides a pharmaceutical composition comprising the enantiomerically enriched γ-amino acid, such as (S)-pregabalin (1), obtained by the novel process of the present invention.

A first aspect of the present invention provides a process of preparing an enantiomerically enriched γ-amino acid, comprising the use of an enantiomer of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, or a derivative thereof, or an enantiomer of a mandelic acid derivative.

Preferably the enantiomerically enriched γ-amino acid obtained is enantiomerically enriched (S)-pregabalin.

Preferably the enantiomer of tartaric acid or a derivative thereof is (L)-tartaric acid, O,O′-di-p-toluoyl-(L)-tartaric acid (including O,O′-di-p-toluoyl-(L)-tartaric acid monohydrate) or O,O′-dibenzoyl-(L)-tartaric acid, preferably (L)-tartaric acid. Preferably the enantiomer of a mandelic acid derivative is (S)-3-chloro-mandelic acid or (S)-3-bromo-mandelic acid.

In a preferred embodiment of the first aspect of the present invention, the process comprises the steps of:

(a) treating a racemic γ-amino acid with an enantiomer of tartaric acid or a derivative thereof to obtain an enantiomerically enriched γ-amino acid salt;
(b) optionally recrystallising the enantiomerically enriched γ-amino acid salt;
(c) dissolving or suspending the enantiomerically enriched γ-amino acid salt obtained in step (a) or (b) in an organic solvent or water or a mixture thereof and adjusting the pH of the solution or suspension with a base to obtain enantiomerically enriched γ-amino acid; and
(d) optionally recrystallising the enantiomerically enriched γ-amino acid.

In another preferred embodiment of the first aspect of the present invention, the process comprises the steps of:

(a) treating racemic pregabalin with (L)-tartaric acid or a derivative thereof to obtain enantiomerically enriched (S)-pregabalin (L)-tartrate or a derivative thereof;
(b) optionally recrystallising the enantiomerically enriched (S)-pregabalin (L)-tartrate or the derivative thereof;
(c) dissolving or suspending the enantiomerically enriched (S)-pregabalin (L)-tartrate or the derivative thereof obtained in step (a) or (b) in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

In another preferred embodiment of the first aspect of the present invention, the process comprises the steps of:

(a) treating a racemic γ-amino acid with an enantiomer of tartaric acid or a derivative thereof to obtain a γ-amino acid salt, wherein the γ-amino acid in the γ-amino acid salt is racemic;
(b) recrystallising the γ-amino acid salt to obtain enantiomerically enriched γ-amino acid salt;
(c) dissolving or suspending the enantiomerically enriched γ-amino acid salt in an organic solvent or water or a mixture thereof and adjusting the pH of the solution or suspension with a base to obtain enantiomerically enriched γ-amino acid; and
(d) optionally recrystallising the enantiomerically enriched γ-amino acid.

In another preferred embodiment of the first aspect of the present invention, the process comprises the steps of:

(a) treating racemic pregabalin with (L)-tartaric acid or a derivative thereof to obtain pregabalin (L)-tartrate or a derivative thereof, wherein the pregabalin in the pregabalin (L)-tartrate or the derivative thereof is racemic;
(b) recrystallising the pregabalin (L)-tartrate or the derivative thereof to obtain enantiomerically enriched (S)-pregabalin (L)-tartrate or a derivative thereof;
(c) dissolving or suspending the enantiomerically enriched (S)-pregabalin (L)-tartrate or the derivative thereof in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

Preferably the enantiomer of tartaric acid or a derivative thereof, used in step (a) of these preferred embodiments, is used in an amount of 0.4 to 10 eq., preferably in an amount of 1 to 1.05 eq., relative to the racemic γ-amino acid used.

Preferably the base used in step (c) of these preferred embodiments is an organic and inorganic base, preferably an organic base. Preferably the organic base is an amine, preferably methylamine, dimethylamine, trimethylamine, ethylamine, diethylamine, triethylamine, N,N-diisopropylethylamine, cyclohexylamine, or a mixture thereof, preferably N,N-diisopropylethylamine. Preferably the inorganic base is ammonia, a metal hydroxide (such as sodium hydroxide, potassium hydroxide or lithium hydroxide), a metal carbonate (such as sodium carbonate, lithium carbonate or calcium carbonate), or a mixture thereof. Preferably the pH of the solution or suspension is adjusted to 4 to 8, preferably 6.5 to 7.5. Preferably step (c) is carried out at a temperature of 0-30° C., preferably 20-30° C.

Preferably step (a) of these preferred embodiments is carried out in an organic solvent in the presence or absence of water. Preferably in step (b) of these preferred embodiments, the enantiomerically enriched γ-amino acid salt is recrystallised from an organic solvent in the presence or absence of water. Step (c) of these preferred embodiments is carried out in an organic solvent or water or a mixture thereof. Preferably in step (d) of these preferred embodiments, the enantiomerically enriched γ-amino acid is recrystallised from an organic solvent or water or a mixture thereof. The organic solvent may be a protic or aprotic solvent. Preferably the organic solvent is an alcohol, a ketone, an ether, an alkane, a cycloalkane, a formamide, an acetate, or a halogenated solvent.

A second aspect of the present invention provides a process of preparing an enantiomerically enriched γ-amino acid salt, comprising crystallising the enantiomerically enriched γ-amino acid salt from a solvent mixture comprising an alcohol and at least 15% water.

Preferably the solvent mixture comprises alcohol:water in a ratio of 85:15 to 50:50, preferably in a ratio of 80:20 to 60:40. Preferably the alcohol is methanol, ethanol, n-propanol, i-propanol, n-butanol, i-butanol, t-butanol, 2-pentanol, 3-pentanol, 4-penten-2-ol, 1,6-hexanediol, 1-hexanol, 5-hexen-1-ol, glycerol, 1-heptanol, 2-heptanol, 1-octanol, 2-octanol, or 3-octanol, preferably n-butanol. A preferred solvent mixture is n-butanol:water in a ratio of 85:15 to 80:20.

Preferably the enantiomerically enriched γ-amino acid salt obtained is an (S)-pregabalin salt.

Preferably the enantiomerically enriched γ-amino acid salt is a salt of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof. More preferably the enantiomerically enriched γ-amino acid salt is a salt of (L)-tartaric acid, O,O′-di-p-toluoyl-(L)-tartaric acid (including O,O′-di-p-toluoyl-(L)-tartaric acid monohydrate), O,O′-dibenzoyl-(L)-tartaric acid, (S)-3-chloro-mandelic acid, or (S)-3-bromo-mandelic acid, preferably (L)-tartaric acid.

In a preferred embodiment of the second aspect of the present invention, the process comprises the step of:

(a) treating a racemic γ-amino acid with an enantiomer of an acid resolving agent in a solvent mixture comprising an alcohol and at least 15% water to obtain an enantiomerically enriched γ-amino acid salt, wherein the enantiomerically enriched γ-amino acid salt crystallises from the solvent mixture.

Preferably the process further comprises the step(s) of:

(b) optionally recrystallising the enantiomerically enriched γ-amino acid salt from a solvent mixture comprising an alcohol and at least 15% water;
(c) dissolving or suspending the enantiomerically enriched γ-amino acid salt obtained in step (a) or (b) in an organic solvent or water or a mixture thereof and adjusting the pH of the solution or suspension with a base to obtain enantiomerically enriched γ-amino acid; and
(d) optionally recrystallising the enantiomerically enriched γ-amino acid.

In another preferred embodiment of the second aspect of the present invention, the process comprises the step of:

(a) treating racemic pregabalin with (L)-tartaric acid or a derivative thereof in a solvent mixture comprising an alcohol and at least 15% water to obtain enantiomerically enriched (S)-pregabalin (L)-tartrate or a derivative thereof, wherein the enantiomerically enriched (S)-pregabalin (L)-tartrate or the derivative thereof crystallises from the solvent mixture.

Preferably the process further comprises the step(s) of:

(b) optionally recrystallising the enantiomerically enriched (S)-pregabalin (L)-tartrate or the derivative thereof from a solvent mixture comprising an alcohol and at least 15% water;
(c) dissolving or suspending the enantiomerically enriched (S)-pregabalin (L)-tartrate or the derivative thereof obtained in step (a) or (b) in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

In another preferred embodiment of the second aspect of the present invention, the process comprises the step of:

(b) recrystallising a γ-amino acid salt, wherein the γ-amino acid in the γ-amino acid salt is racemic, from a solvent mixture comprising an alcohol and at least 15% water to obtain enantiomerically enriched γ-amino acid salt.

Preferably step (b) is preceded by the step of:

(a) treating a racemic γ-amino acid with an enantiomer of an acid resolving agent to obtain the γ-amino acid salt.

Preferably the process further comprises the step(s) of:

(c) dissolving or suspending the enantiomerically enriched γ-amino acid salt in an organic solvent or water or a mixture thereof and adjusting the pH of the solution or suspension with a base to obtain enantiomerically enriched γ-amino acid; and
(d) optionally recrystallising the enantiomerically enriched γ-amino acid.

In another preferred embodiment of the second aspect of the present invention, the process comprises the step of:

(b) recrystallising pregabalin (L)-tartrate or a derivative thereof, wherein the pregabalin in the pregabalin (L)-tartrate or the derivative thereof is racemic, from a solvent mixture comprising an alcohol and at least 15% water to obtain enantiomerically enriched (S)-pregabalin (L)-tartrate or a derivative thereof.

Preferably step (b) is preceded by the step of:

(a) treating racemic pregabalin with (L)-tartaric acid or a derivative thereof to obtain the pregabalin (L)-tartrate or the derivative thereof.

Preferably the process further comprises the step(s) of:

(c) dissolving or suspending the enantiomerically enriched (S)-pregabalin (L)-tartrate or the derivative thereof in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

Preferably the acid resolving agent is tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof. More preferably the acid resolving agent is (L)-tartaric acid, O,O′-di-p-toluoyl-(L)-tartaric acid (including O,O′-di-p-toluoyl-(L)-tartaric acid monohydrate), O,O′-dibenzoyl-(L)-tartaric acid, (S)-3-chloro-mandelic acid, or (S)-3-bromo-mandelic acid, preferably (L)-tartaric acid. Preferably the acid resolving agent, used in step (a) of these preferred embodiments, is used in an amount of 0.4 to 10 eq., preferably in an amount of 1 to 1.05 eq., relative to the racemic γ-amino acid used.

Preferably the base used in step (c) of these preferred embodiments is an organic and inorganic base, preferably an organic base. Preferably the organic base is an amine, preferably methylamine, dimethylamine, trimethylamine, ethylamine, diethylamine, triethylamine, N,N-diisopropylethylamine, cyclohexylamine, or a mixture thereof, preferably N,N-diisopropylethylamine. Preferably the inorganic base is ammonia, a metal hydroxide (such as sodium hydroxide, potassium hydroxide or lithium hydroxide), a metal carbonate (such as sodium carbonate, lithium carbonate or calcium carbonate), or a mixture thereof. Preferably the pH of the solution or suspension is adjusted to 4 to 8, preferably 6.5 to 7.5. Preferably step (c) is carried out at a temperature of 0-30° C., preferably 20-30° C.

Step (c) of these preferred embodiments is carried out in an organic solvent or water or a mixture thereof. Preferably in step (d) of these preferred embodiments, the enantiomerically enriched γ-amino acid is recrystallised from an organic solvent or water or a mixture thereof. The organic solvent may be a protic or aprotic solvent. Preferably the organic solvent is an alcohol, a ketone, an ether, an alkane, a cycloalkane, a formamide, an acetate, or a halogenated solvent.

A third aspect of the present invention provides a process of preparing an enantiomerically enriched γ-amino acid, comprising the step of:

(c) dissolving or suspending an enantiomerically enriched γ-amino acid salt in an organic solvent or water or a mixture thereof and adjusting the pH of the solution or suspension with a base to obtain enantiomerically enriched γ-amino acid.

Preferably the enantiomerically enriched γ-amino acid salt is an (S)-pregabalin salt, and the enantiomerically enriched γ-amino acid is (S)-pregabalin.

Preferably the enantiomerically enriched γ-amino acid salt is a salt of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof. More preferably the enantiomerically enriched γ-amino acid salt is a salt of (L)-tartaric acid, O,O′-di-p-toluoyl-(L)-tartaric acid (including O,O′-di-p-toluoyl-(L)-tartaric acid monohydrate), O,O′-dibenzoyl-(L)-tartaric acid, (S)-3-chloro-mandelic acid, or (S)-3-bromo-mandelic acid, preferably (L)-tartaric acid.

In a preferred embodiment of the third aspect of the present invention, step (c) is preceded by the step(s) of:

(a) treating a racemic γ-amino acid with an enantiomer of an acid resolving agent to obtain an enantiomerically enriched γ-amino acid salt; and
(b) optionally recrystallising the enantiomerically enriched γ-amino acid salt.

In another preferred embodiment of the third aspect of the present invention, step (c) is preceded by the steps of:

(a) treating a racemic γ-amino acid with an enantiomer of an acid resolving agent to obtain a γ-amino acid salt, wherein the γ-amino acid in the γ-amino acid salt is racemic; and
(b) recrystallising the γ-amino acid salt to obtain enantiomerically enriched γ-amino acid salt.

Preferably the acid resolving agent is tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof. More preferably the acid resolving agent is (L)-tartaric acid, O,O′-di-p-toluoyl-(L)-tartaric acid (including O,O′-di-p-toluoyl-(L)-tartaric acid monohydrate), O,O′-dibenzoyl-(L)-tartaric acid, (S)-3-chloro-mandelic acid, or (S)-3-bromo-mandelic acid, preferably (L)-tartaric acid. Preferably the acid resolving agent, used in step (a) of these preferred embodiments, is used in an amount of 0.4 to 10 eq., preferably in an amount of 1 to 1.05 eq., relative to the racemic γ-amino acid used.

Preferably the process further comprises the step of:

(d) recrystallising the enantiomerically enriched γ-amino acid.

In another preferred embodiment of the third aspect of the present invention, the process comprises the step of:

(c) dissolving or suspending enantiomerically enriched (S)-pregabalin (L)-tartrate or a derivative thereof in an organic solvent or water or a mixture thereof and adjusting the pH of the solution or suspension with a base to obtain enantiomerically enriched (S)-pregabalin or a derivative thereof.

Preferably this step (c) is preceded by the step(s) of:

(a) treating racemic pregabalin with (L)-tartaric acid or a derivative thereof to obtain enantiomerically enriched (S)-pregabalin (L)-tartrate or a derivative thereof; and
(b) optionally recrystallising the enantiomerically enriched (S)-pregabalin (L)-tartrate or the derivative thereof.

Alternatively, this step (c) is preferably preceded by the steps of:

(a) treating racemic pregabalin with (L)-tartaric acid or a derivative thereof to obtain pregabalin (L)-tartrate or a derivative thereof, wherein the pregabalin in the pregabalin (L)-tartrate or the derivative thereof is racemic; and
(b) recrystallising the pregabalin (L)-tartrate or the derivative thereof to obtain enantiomerically enriched (S)-pregabalin (L)-tartrate or a derivative thereof.

Preferably the process further comprises the step of:

(d) recrystallising the enantiomerically enriched (S)-pregabalin.

Preferably the base used in step (c) is an organic and inorganic base, preferably an organic base. Preferably the organic base is an amine, preferably methylamine, dimethylamine, trimethylamine, ethylamine, diethylamine, triethylamine, N,N-diisopropylethylamine, cyclohexylamine, or a mixture thereof, preferably N,N-diisopropylethylamine. Preferably the inorganic base is ammonia, a metal hydroxide (such as sodium hydroxide, potassium hydroxide or lithium hydroxide), a metal carbonate (such as sodium carbonate, lithium carbonate or calcium carbonate), or a mixture thereof. Preferably the pH of the solution or suspension is adjusted to 4 to 8, preferably 6.5 to 7.5. Preferably step (c) is carried out at a temperature of 0-30° C., preferably 20-30° C.

Preferably step (a) of these preferred embodiments is carried out in an organic solvent in the presence or absence of water. Preferably in step (b) of these preferred embodiments, the enantiomerically enriched γ-amino acid salt is recrystallised from an organic solvent in the presence or absence of water. Step (c) of these preferred embodiments is carried out in an organic solvent or water or a mixture thereof. Preferably in step (d) of these preferred embodiments, the enantiomerically enriched γ-amino acid is recrystallised from an organic solvent or water or a mixture thereof. The organic solvent may be a protic or aprotic solvent. Preferably the organic solvent is an alcohol, a ketone, an ether, an alkane, a cycloalkane, a formamide, an acetate, or a halogenated solvent.

In all embodiments of the process of the present invention, preferably none of the reaction steps involves seeding.

In all embodiments of the process of the present invention, preferably the enantiomerically enriched γ-amino acid is prepared on an industrial scale, preferably in batches of 0.5 kg, 1 kg, 10 kg, 50 kg, 100 kg, 500 kg or more.

In all embodiments of the process of the present invention, preferably the reaction temperature throughout the reaction is less than 80° C., preferably less than 70° C., preferably less than 60° C., preferably less than 50° C.

In all embodiments of the process of the present invention, preferably the total reaction time is less than 30 hours, preferably less than 25 hours, preferably less than 20 hours, preferably less than 15 hours.

In all embodiments of the process of the present invention, preferably the enantiomerically enriched γ-amino acid salt is obtained in a molar yield of 80%, 85%, 90%, 95% or more. Preferably the enantiomerically enriched γ-amino acid salt obtained has an enantiomeric purity of 95%, 98%, 99%, 99.5%, 99.9% or more (as measured by chiral HPLC). Preferably the enantiomerically enriched γ-amino acid salt obtained has a chemical purity of 95%, 98%, 99%, 99.5%, 99.9% or more (as measured by HPLC). Preferably the enantiomerically enriched γ-amino acid salt obtained comprises 3%, 2%, 1%, 0.5%, 0.1% or less of the corresponding lactam (as measured by HPLC).

In all embodiments of the process of the present invention, preferably the enantiomerically enriched γ-amino acid is obtained in a molar yield of 80%, 85%, 90%, 95% or more. Preferably the enantiomerically enriched γ-amino acid obtained has an enantiomeric purity of 99%, 99.5%, 99.9%, 99.99% or more (as measured by chiral HPLC). Preferably the enantiomerically enriched γ-amino acid obtained has a chemical purity of 99%, 99.5%, 99.9%, 99.99% or more (as measured by HPLC). Preferably the enantiomerically enriched γ-amino acid obtained comprises 1%, 0.5%, 0.1%, 0.01% or less of the corresponding lactam (as measured by HPLC).

A fourth aspect of the present invention provides an enantiomerically enriched γ-amino acid obtained by the process of the first, second and third aspect of the present invention. The fourth aspect of the present invention also provides a γ-amino acid having an enantiomeric purity of 99%, 99.5%, 99.9%, 99.99% or more (as measured by chiral HPLC). The fourth aspect of the present invention also provides a γ-amino acid having a chemical purity of 99%, 99.5%, 99.9%, 99.99% or more (as measured by HPLC). The fourth aspect of the present invention also provides a γ-amino acid salt having an enantiomeric purity of 95%, 98%, 99%, 99.5%, 99.9% or more (as measured by chiral HPLC). The fourth aspect of the present invention also provides a γ-amino acid salt having a chemical purity of 95%, 98%, 99%, 99.5%, 99.9% or more (as measured by HPLC).

A fifth aspect of the present invention provides a pharmaceutical composition comprising the γ-amino acid or the γ-amino acid salt of the fourth aspect of the present invention.

### DETAILED DESCRIPTION OF THE INVENTION

The process of the present invention has the following advantages:
The resolving agent used, preferably an enantiomer of tartaric acid or a derivative thereof, is inexpensive and easily available.
The process can be carried out under mild reaction conditions.
None of the process steps takes longer than about 4 hours, therefore the overall process is quick.
A high yield of resolution is achieved (32% w/w compared to the prior art resolution of 27% w/w).
An excellent enantiomeric purity is achieved in the resolution step (98-99%).
The process is convenient and easy to carry out.
The isolation of the enantiomerically enriched γ-amino acid is convenient.
The enantiomerically enriched γ-amino acid obtained is of high enough quality to meet the requirements of the ICH guidelines.
Acid or heat catalysed lactam formation is minimised.
The process is suitable for commercial scale manufacture.

The present invention provides a simple, convenient, and inexpensive process of preparing enantiomerically enriched (S)-pregabalin (1), which is widely used as anticonvulsant agent. A preferred embodiment of the present invention is a process of resolving racemic pregabalin (2) using (L)-tartaric acid or a derivative thereof to obtain enantiomerically enriched (S)-pregabalin (1).

A first preferred embodiment of the process of the present invention is outlined in Scheme 2.

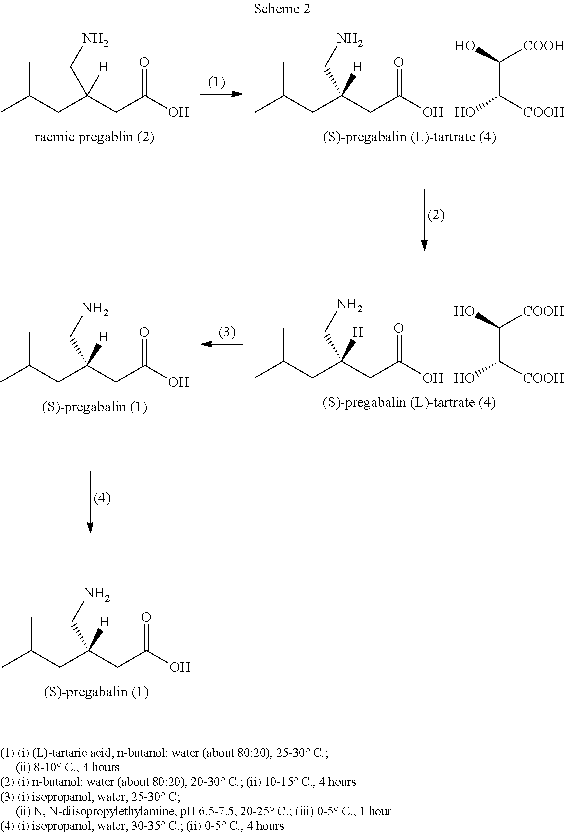

This preferred embodiment comprises three or four steps: (1) a resolution step, (2) a fractional crystallisation step, (3) an isolation step, and optionally (4) a purification step. In the resolution step, racemic pregabalin (2) is treated with (L)-tartaric acid to obtain enantiomerically enriched (S)-pregabalin (L)-tartrate (4) (98-99% S-isomer as measured by chiral HPLC). In the fractional crystallisation step, the fractional crystallisation of the (S)-pregabalin (L)-tartrate (4) increases its enantiomeric purity to 99.75-100% S-isomer (as measured by chiral HPLC). In the isolation step, (S)-pregabalin (1) is isolated from the enantiomerically enriched (S)-pregabalin (L)-tartrate (4). In the optional purification step, (S)-pregabalin (1) is purified by recrystallisation.

In step (1), the racemic pregabalin (2) is preferably dissolved or suspended in an organic protic or aprotic solvent, in a mixture of organic protic or aprotic solvents, in water, or in a mixture of one or more organic protic or aprotic solvents and water. Preferably the organic protic or aprotic solvent is an alcohol, a ketone, an ether, an alkane, a cycloalkane, a formamide, an acetate, or a halogenated solvent. A preferred solvent is a mixture of an alcohol (preferably n-butanol) and water, preferably comprising 0.5% to 50% water, preferably comprising 15% to 25% water. The racemic pregabalin (2) is preferably dissolved or suspended at a temperature of 5-30° C., preferably 20-25° C.

(L)-Tartaric acid is preferably used in an amount of 0.4 eq. to 10 eq. relative to racemic pregabalin (2), preferably 1 to 1.05 eq. (L)-Tartaric acid may be added to a solution or suspension of racemic pregabalin (2) either as a solid or in solution. A preferred solution is (L)-tartaric acid in an organic protic or aprotic solvent, in a mixture of organic protic or aprotic solvents, in water, or in a mixture of one or more organic protic or aprotic solvents and water. Preferably the organic protic or aprotic solvent is an alcohol, a ketone, an ether, an alkane, a cycloalkane, a formamide, an acetate, or a halogenated solvent. Preferably (L)-tartaric acid is added to a solution or suspension of racemic pregabalin (2) as a solid. The preferred temperature for the addition of (L)-tartaric acid is 5-30° C., preferably 20-25° C.

After the addition of (L)-tartaric acid, the reaction mixture is preferably stirred at a temperature of 0-70° C., preferably 10-30° C., preferably 25-30° C., to obtain a clear solution. The clear solution is preferably stirred for a period of 5 minutes to 10 hours, preferably about 1 hour. Then the clear solution is preferably filtered through a bed of Celite®. The filtrate obtained is preferably stirred at a temperature of −10 to 30° C. for 5 minutes to 10 hours, preferably at 8-15° C. for about 4 hours, to precipitate out enantiomerically enriched (S)-pregabalin (L)-tartrate (4).

The (S)-pregabalin (L)-tartrate (4) is preferably isolated by filtration and preferably dried at a temperature of 40-60° C., preferably 40-45° C. The enantiomeric purity of the (S)-pregabalin (L)-tartrate (4) obtained is preferably 95%, 98%, 98.5%, 99% or more (as measured by chiral HPLC). The molar yield of the (S)-pregabalin (L)-tartrate (4) obtained is preferably 80%, 90%, 95% or more.

In step (2), the (S)-pregabalin (L)-tartrate (4) is subjected to fractional crystallisation to improve its enantiomeric purity. The (S)-pregabalin (L)-tartrate (4) is preferably dissolved or suspended in an organic protic or aprotic solvent, in a mixture of organic protic or aprotic solvents, in water, or in a mixture of one or more organic protic or aprotic solvents and water. Preferably the organic protic or aprotic solvent is an alcohol, a ketone, an ether, an alkane, a cycloalkane, a formamide, an acetate, or a halogenated solvent. A preferred solvent is a mixture of an alcohol (preferably n-butanol) and water, preferably comprising 0.5% to 50% water, preferably comprising 15% to 25% water. The (S)-pregabalin (L)-tartrate (4) is preferably dissolved or suspended at a temperature of 5-30° C., preferably 20-25° C.

The solution or suspension of (S)-pregabalin (L)-tartrate (4) is preferably stirred at a temperature of 0-30° C., preferably 20-30° C., to obtain a clear solution. The clear solution is preferably stirred for a period of 5 minutes to 10 hours, preferably about 1 hour. Then the clear solution is preferably filtered through a bed of Celite®. The filtrate obtained is preferably stirred at a temperature of −10 to 30° C. for 5 minutes to 10 hours, preferably at 10-15° C. for about 4 hours, to precipitate out (S)-pregabalin (L)-tartrate (4) with a greater enantiomeric purity.

The (S)-pregabalin (L)-tartrate (4) is preferably isolated by filtration and preferably dried at a temperature of 40-60° C., preferably 40-45° C. The enantiomeric purity of the (S)-pregabalin (L)-tartrate (4) obtained is preferably 99%, 99.5%, 99.8%, 99.9%, 99.99% or more (as measured by chiral HPLC). The molar yield of the (S)-pregabalin (L)-tartrate (4) obtained is preferably 90%, 95% or more.

In step (3), (S)-pregabalin (1) is isolated from the (S)-pregabalin (L)-tartrate (4). The (S)-pregabalin (L)-tartrate (4) is preferably dissolved or suspended in an organic protic or aprotic solvent, in a mixture of organic protic or aprotic solvents, in water, or in a mixture of one or more organic protic or aprotic solvents and water. Preferably the organic protic or aprotic solvent is an alcohol, a ketone, an ether, an alkane, a cycloalkane, a formamide, an acetate, or a halogenated solvent. A preferred solvent is a mixture of an alcohol (preferably isopropanol) and water, preferably comprising 0.5% to 50% water, preferably comprising 10% to 25% water. Another preferred solvent is a mixture of a ketone and water, preferably comprising 0.5% to 50% water, preferably comprising 10% to 25% water. Another preferred solvent is one organic protic or aprotic solvent without any other solvents. Another preferred solvent is water without any other solvents. The (S)-pregabalin (L)-tartrate (4) is preferably dissolved or suspended at a temperature of 5-30° C., preferably 20-25° C.

In order to isolate the (S)-pregabalin (1) from the (S)-pregabalin (L)-tartrate (4), preferably the pH of the solution or suspension of the (S)-pregabalin (L)-tartrate (4) is adjusted to 4 to 8, preferably 6.5 to 7.5. The pH is preferably adjusted by using an organic and inorganic base, preferably an organic base. Preferred organic bases are methylamine, dimethylamine, trimethylamine, ethylamine, diethylamine, triethylamine, N,N-diisopropylethylamine, or cyclohexylamine, preferably N,N-diisopropylethylamine. Preferred inorganic bases are ammonia; a metal hydroxide such as sodium hydroxide, potassium hydroxide, lithium hydroxide; a metal carbonate such as sodium carbonate, lithium carbonate, calcium carbonate; and mixtures thereof. The pH is preferably adjusted at a temperature of −10 to 30° C., preferably 20-25° C. The reaction mixture is preferably stirred for 5 minutes to 6 hours preferably at a temperature of −10 to 30° C., preferably for 1-2 hours preferably at a temperature of 0-5° C.

The (S)-pregabalin (1) is preferably isolated by filtration and preferably dried at a temperature of 35-60° C., preferably 40-45° C. The enantiomeric purity of the (S)-pregabalin (1) obtained is preferably 99.8%, 99.9%, 99.99% or more (as measured by chiral HPLC). The chemical purity of the (S)-pregabalin (1) obtained is preferably 98.5%, 99%, 99.5% or more (as measured by HPLC). The molar yield of the (S)-pregabalin (1) obtained is preferably 85%, 90% or more.

In optional step (4), the (S)-pregabalin (1) is optionally further purified by recrystallisation, preferably from an organic protic or aprotic solvent, from a mixture of organic protic or aprotic solvents, from water, or from a mixture of one or more organic protic or aprotic solvents and water. Preferably the organic protic or aprotic solvent is an alcohol, a ketone, an ether, an alkane, a cycloalkane, a formamide, an acetate, or a halogenated solvent. A preferred solvent is a mixture of an alcohol (preferably isopropanol) and water, preferably comprising 0.5% to 50% water, preferably comprising 10% to 25% water.

Preferably the (S)-pregabalin (1) is dissolved or suspended, and the solution or suspension is preferably stirred at a temperature of 30-35° C. preferably for 0.5-3 hours to obtain a clear solution. Preferably the reaction mixture is cooled to a temperature of 0-5° C. preferably for 2-4 hours to precipitate out the (S)-pregabalin (1).

The (S)-pregabalin (1) is preferably isolated by filtration and preferably dried at a temperature of 35-60° C., preferably 40-45° C. The enantiomeric purity of the (S)-pregabalin (1) obtained is preferably 99.9%, 99.99% or more (as measured by chiral HPLC). The chemical purity of the (S)-pregabalin (1) obtained is preferably 99%, 99.5% or more (as measured by HPLC). The molar yield of the (S)-pregabalin (1) obtained is preferably 85%, 90% or more.

Preferably none of the four steps described above involves seeding.

By following reaction conditions similar to those mentioned above, resolution of racemic pregabalin (2) to obtain (S)-pregabalin (1) can also be achieved using an enantiomer of a tartaric acid derivative (such as O,O′-di-p-toluoyl-(L)-tartaric acid (including O,O′-di-p-toluoyl-(L)-tartaric acid monohydrate) or O,O′-dibenzoyl-(L)-tartaric acid), or an enantiomer of mandelic acid or a derivative thereof (such as (S)-3-chloro-mandelic acid or (S)-3-bromo-mandelic acid), or an enantiomer of camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, or a derivative thereof.

By following reaction conditions similar to those mentioned above, resolution of racemic γ-amino acids other than racemic pregabalin (2) can also be achieved.

A second preferred embodiment of the process of the present invention is outlined in Scheme 3.

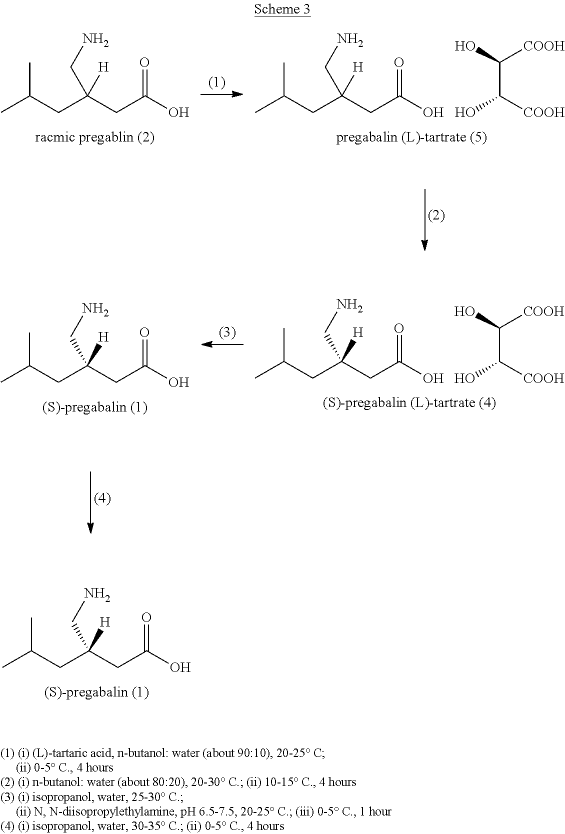

This preferred embodiment comprises three or four steps: (1) a salt preparation step, (2) a fractional crystallisation step, (3) an isolation step, and optionally (4) a purification step. In the salt preparation step, racemic pregabalin (2) is treated with (L)-tartaric acid to obtain pregabalin (L)-tartrate (5), wherein the pregabalin in the pregabalin (L)-tartrate (5) is racemic. In the fractional crystallisation step, the pregabalin (L)-tartrate (5) is fractionally crystallised to obtain enantiomerically enriched (S)-pregabalin (L)-tartrate (4) with an enantiomeric purity of 99.75-99.95% S-isomer (as measured by chiral HPLC). In the isolation step, (S)-pregabalin (1) is isolated from the enantiomerically enriched (S)-pregabalin (L)-tartrate (4). In the optional purification step, (S)-pregabalin (1) is purified by recrystallisation.

In step (1), pregabalin (L)-tartrate salt (5) is prepared using (L)-tartaric acid. Racemic pregabalin (2) is preferably dissolved or suspended in an organic protic or aprotic solvent, a mixture of organic protic or aprotic solvents, water, or a mixture of one or more organic protic or aprotic solvents and water. Preferably the organic protic or aprotic solvent is an alcohol, a ketone, an ether, an alkane, a cycloalkane, a formamide, an acetate, or a halogenated solvent. A preferred solvent is a mixture of an alcohol (preferably n-butanol) and water, preferably comprising 0.5% to 50% water, preferably comprising 5% to 12% water. The racemic pregabalin (2) is preferably dissolved or suspended at a temperature of 5-30° C., preferably 20-25° C.

(L)-Tartaric acid is preferably used in an amount of 0.4 eq. to 10 eq. relative to racemic pregabalin (2), preferably 1 to 1.05 eq. (L)-Tartaric acid may be added to a solution or suspension of racemic pregabalin (2) either as a solid or in solution. A preferred solution is (L)-tartaric acid in an organic protic or aprotic solvent, in a mixture of organic protic or aprotic solvents, in water, or in a mixture of one or more organic protic or aprotic solvents and water. Preferably the organic protic or aprotic solvent is an alcohol, a ketone, an ether, an alkane, a cycloalkane, a formamide, an acetate, or a halogenated solvent. Preferably (L)-tartaric acid is added to a solution or suspension of racemic pregabalin (2) as a solid. The preferred temperature for the addition of (L)-tartaric acid is 5-30° C., preferably 20-25° C.

After the addition of (L)-tartaric acid, the reaction mixture is preferably stirred at a temperature of 0-70° C., preferably 10-30° C., preferably 25-30° C., to obtain a clear solution. The clear solution is preferably stirred for a period of 5 minutes to 10 hours, preferably for about 1 hour, at a temperature of 20-25° C. The clear solution is preferably further stirred for a period of 5 minutes to 10 hours at a temperature of −10 to 10° C., preferably for about 4 hours at 0-5° C., to precipitate out pregabalin (L)-tartrate (5), wherein the pregabalin in the pregabalin (L)-tartrate (5) is racemic.

The pregabalin (L)-tartrate (5) is preferably isolated by filtration and preferably dried at a temperature of 40-60° C., preferably 40-45° C. The molar yield of the pregabalin (L)-tartrate (5) obtained is preferably 80%, 90%, 95%, 98% or more.

In step (2), the pregabalin (L)-tartrate (5) is subjected to fractional crystallisation to obtain enantiomerically enriched (S)-pregabalin (L)-tartrate (4). The pregabalin (L)-tartrate (5) is preferably dissolved or suspended in an organic protic or aprotic solvent, in a mixture of organic protic or aprotic solvents, in water, or in a mixture of one or more organic protic or aprotic solvents and water. Preferably the organic protic or aprotic solvent is an alcohol, a ketone, an ether, an alkane, a cycloalkane, a formamide, an acetate, or a halogenated solvent. A preferred solvent is a mixture of an alcohol (preferably n-butanol) and water, preferably comprising 0.5% to 50% water, preferably comprising 15% to 25% water. The pregabalin (L)-tartrate (5) is preferably dissolved or suspended at a temperature of 5-30° C., preferably 20-25° C.

The solution or suspension of pregabalin (L)-tartrate (5) is preferably stirred at a temperature of 0-30° C., preferably 20-30° C., to obtain a clear solution. The clear solution is preferably stirred for a period of 5 minutes to 10 hours, preferably about 1 hour. Then the clear solution is preferably filtered through a bed of Celite®. The filtrate obtained is preferably stirred at a temperature of −10 to 30° C. for 5 minutes to 10 hours, preferably at 10-15° C. for about 4 hours, to precipitate out enantiomerically enriched (S)-pregabalin (L)-tartrate (4).

The enantiomerically enriched (S)-pregabalin (L)-tartrate (4) is preferably isolated by filtration and preferably dried at a temperature of 40-60° C., preferably 40-45° C. The enantiomeric purity of the enantiomerically enriched (S)-pregabalin (L)-tartrate (4) obtained is preferably 99%, 99.5%, 99.8%, 99.9%, 99.99% or more (as measured by chiral HPLC). The molar yield of the enantiomerically enriched (S)-pregabalin (L)-tartrate (4) obtained is preferably 80%, 85%, 90%, 95% or more.

Steps (3) and (4) are carried out as described in relation to Scheme 2.

Preferably none of the four steps described above involves seeding.

By following reaction conditions similar to those mentioned above, resolution of racemic pregabalin (2) to obtain (S)-pregabalin (1) can also be achieved using an enantiomer of a tartaric acid derivative (such as O,O′-di-p-toluoyl-(L)-tartaric acid (including O,O′-di-p-toluoyl-(L)-tartaric acid monohydrate) or O,O′-dibenzoyl-(L)-tartaric acid), or an enantiomer of mandelic acid or a derivative thereof (such as (S)-3-chloro-mandelic acid or (S)-3-bromo-mandelic acid), or an enantiomer of camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, or a derivative thereof.

By following reaction conditions similar to those mentioned above, resolution of racemic γ-amino acids other than racemic pregabalin (2) can also be achieved.

The following paragraphs enumerated consecutively from 1 through 89 provide for various aspects of the present invention. In one embodiment, the present invention provides:

1. A process of preparing an enantiomerically enriched γ-amino acid, comprising the use of an enantiomer of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, or a derivative thereof, or an enantiomer of a mandelic acid derivative.
2. The process of paragraph 1, wherein the enantiomerically enriched γ-amino acid is enantiomerically enriched (S)-pregabalin.
3. The process of paragraph 1 or 2, wherein the enantiomer of tartaric acid or a derivative thereof is (L)-tartaric acid, O,O′-di-p-toluoyl-(L)-tartaric acid or O,O′-dibenzoyl-(L)-tartaric acid.
4. The process of paragraph 3, wherein the enantiomer of tartaric acid or a derivative thereof is (L)-tartaric acid.
5. The process of paragraph 1 or 2, wherein the enantiomer of a mandelic acid derivative is (S)-3-chloro-mandelic acid or (S)-3-bromo-mandelic acid.
6. The process of any one of paragraphs 1 to 5, comprising the steps of:
(a) treating a racemic γ-amino acid with an enantiomer of tartaric acid or a derivative thereof to obtain an enantiomerically enriched γ-amino acid salt;
(b) optionally recrystallising the enantiomerically enriched γ-amino acid salt;
(c) dissolving or suspending the enantiomerically enriched γ-amino acid salt obtained in step (a) or (b) in an organic solvent or water or a mixture thereof and adjusting the pH of the solution or suspension with a base to obtain enantiomerically enriched γ-amino acid; and
(d) optionally recrystallising the enantiomerically enriched γ-amino acid.
7. The process of paragraph 6, comprising the steps of:
(a) treating racemic pregabalin with (L)-tartaric acid or a derivative thereof to obtain enantiomerically enriched (S)-pregabalin (L)-tartrate or a derivative thereof;
(b) optionally recrystallising the enantiomerically enriched (S)-pregabalin (L)-tartrate or the derivative thereof;
(c) dissolving or suspending the enantiomerically enriched (S)-pregabalin (L)-tartrate or the derivative thereof obtained in step (a) or (b) in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.
8. The process of any one of paragraphs 1 to 5, comprising the steps of:
(a) treating a racemic γ-amino acid with an enantiomer of tartaric acid or a derivative thereof to obtain a γ-amino acid salt, wherein the γ-amino acid in the γ-amino acid salt is racemic;
(b) recrystallising the γ-amino acid salt to obtain enantiomerically enriched γ-amino acid salt;
(c) dissolving or suspending the enantiomerically enriched γ-amino acid salt in an organic solvent or water or a mixture thereof and adjusting the pH of the solution or suspension with a base to obtain enantiomerically enriched γ-amino acid; and
(d) optionally recrystallising the enantiomerically enriched γ-amino acid.
9. The process of paragraph 8, comprising the steps of:
(a) treating racemic pregabalin with (L)-tartaric acid or a derivative thereof to obtain pregabalin (L)-tartrate or a derivative thereof, wherein the pregabalin in the pregabalin (L)-tartrate or the derivative thereof is racemic;
(b) recrystallising the pregabalin (L)-tartrate or the derivative thereof to obtain enantiomerically enriched (S)-pregabalin (L)-tartrate or a derivative thereof;
(c) dissolving or suspending the enantiomerically enriched (S)-pregabalin (L)-tartrate or the derivative thereof in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.
10. The process of any one of paragraphs 6 to 9, wherein the base used in step (c) is an organic and inorganic base.
11. The process of paragraph 10, wherein the organic base is an amine.
12. The process of paragraph 11, wherein the amine is methylamine, dimethylamine, trimethylamine, ethylamine, diethylamine, triethylamine, N,N-diisopropylethylamine, cyclohexylamine, or a mixture thereof.
13. The process of paragraph 10, wherein the inorganic base is ammonia, a metal hydroxide, a metal carbonate, or a mixture thereof.
14. The process of paragraph 13, wherein the metal hydroxide is sodium hydroxide, potassium hydroxide or lithium hydroxide, and/or wherein the metal carbonate is sodium carbonate, lithium carbonate or calcium carbonate.
15. The process of any one of paragraphs 6 to 14, wherein step (a) is carried out in an organic solvent in the presence or absence of water.
16. The process of any one of paragraphs 6 to 15, wherein in step (b) the enantiomerically enriched γ-amino acid salt is recrystallised from an organic solvent in the presence or absence of water.
17. The process of any one of paragraphs 6 to 16, wherein in step (d) the enantiomerically enriched γ-amino acid is recrystallised from an organic solvent or water or a mixture thereof.
18. The process of any one of paragraphs 6 to 17, wherein the organic solvent is a protic or aprotic solvent.
19. The process of any one of paragraphs 6 to 18, wherein the organic solvent is an alcohol, a ketone, an ether, an alkane, a cycloalkane, a formamide, an acetate, or a halogenated solvent.
20. A process of preparing an enantiomerically enriched γ-amino acid salt, comprising crystallising the enantiomerically enriched γ-amino acid salt from a solvent mixture comprising an alcohol and at least 15% water.
21. The process of paragraph 20, wherein the solvent mixture comprises alcohol:water in a ratio of 85:15 to 50:50.
22. The process of paragraph 21, wherein the solvent mixture comprises alcohol:water in a ratio of 80:20 to 60:40.
23. The process of any one of paragraphs 20 to 22, wherein the alcohol is methanol, ethanol, n-propanol, i-propanol, n-butanol, i-butanol, t-butanol, 2-pentanol, 3-pentanol, 4-penten-2-ol, 1,6-hexanediol, 1-hexanol, 5-hexen-1-ol, glycerol, 1-heptanol, 2-heptanol, 1-octanol, 2-octanol, or 3-octanol.
24. The process of paragraph 23, wherein the alcohol is n-butanol.
25. The process of any one of paragraphs 20 to 24, wherein the enantiomerically enriched γ-amino acid salt is an (S)-pregabalin salt.
26. The process of any one of paragraphs 20 to 25, wherein the enantiomerically enriched γ-amino acid salt is a salt of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-β-isopropylidene-2-oxo-L-gulonic acid, 2-hydroxy-4-isopropenyl-1-methyl-cyclohexane-1-sulphonic acid, mandelic acid, or a derivative thereof.
27. The process of paragraph 26, wherein the enantiomerically enriched γ-amino acid salt is a salt of (L)-tartaric acid, O,O′-di-p-toluoyl-(L)-tartaric acid, O,O′-dibenzoyl-(L)-tartaric acid, (S)-3-chloro-mandelic acid, or (S)-3-bromo-mandelic acid.
28. The process of paragraph 27, wherein the enantiomerically enriched γ-amino acid salt is a salt of (L)-tartaric acid.
29. The process of any one of paragraphs 20 to 28, comprising the step of:
(a) treating a racemic γ-amino acid with an enantiomer of an acid resolving agent in a solvent mixture comprising an alcohol and at least 15% water to obtain an enantiomerically enriched γ-amino acid salt, wherein the enantiomerically enriched γ-amino acid salt crystallises from the solvent mixture.
30. The process of paragraph 29, further comprising the step(s) of:
(b) optionally recrystallising the enantiomerically enriched γ-amino acid salt from a solvent mixture comprising an alcohol and at least 15% water;
(c) dissolving or suspending the enantiomerically enriched γ-amino acid salt obtained in step (a) or (b) in an organic solvent or water or a mixture thereof and adjusting the pH of the solution or suspension with a base to obtain enantiomerically enriched γ-amino acid; and
(d) optionally recrystallising the enantiomerically enriched γ-amino acid.
31. The process of paragraph 29, comprising the step of:
(a) treating racemic pregabalin with (L)-tartaric acid or a derivative thereof in a solvent mixture comprising an alcohol and at least 15% water to obtain enantiomerically enriched (S)-pregabalin (L)-tartrate or a derivative thereof, wherein the enantiomerically enriched (S)-pregabalin (L)-tartrate or the derivative thereof crystallises from the solvent mixture.
32. The process of paragraph 31, further comprising the step(s) of:
(b) optionally recrystallising the enantiomerically enriched (S)-pregabalin (L)-tartrate or the derivative thereof from a solvent mixture comprising an alcohol and at least 15% water;
(c) dissolving or suspending the enantiomerically enriched (S)-pregabalin (L)-tartrate or the derivative thereof obtained in step (a) or (b) in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.
33. The process of any one of paragraphs 20 to 28, comprising the step of:
(b) recrystallising a γ-amino acid salt, wherein the γ-amino acid in the γ-amino acid salt is racemic, from a solvent mixture comprising an alcohol and at least 15% water to obtain enantiomerically enriched γ-amino acid salt.
34. The process of paragraph 33, wherein step (b) is preceded by the step of:
(a) treating a racemic γ-amino acid with an enantiomer of an acid resolving agent to obtain the γ-amino acid salt.
35. The process of paragraph 33 or 34, further comprising the step(s) of:
(c) dissolving or suspending the enantiomerically enriched γ-amino acid salt in an organic solvent or water or a mixture thereof and adjusting the pH of the solution or suspension with a base to obtain enantiomerically enriched γ-amino acid; and
(d) optionally recrystallising the enantiomerically enriched γ-amino acid.
36. The process of paragraph 33, comprising the step of:
(b) recrystallising pregabalin (L)-tartrate or a derivative thereof, wherein the pregabalin in the pregabalin (L)-tartrate or the derivative thereof is racemic, from a solvent mixture comprising an alcohol and at least 15% water to obtain enantiomerically enriched (S)-pregabalin (L)-tartrate or a derivative thereof.
37. The process of paragraph 36, wherein step (b) is preceded by the step of:
(a) treating racemic pregabalin with (L)-tartaric acid or a derivative thereof to obtain the pregabalin (L)-tartrate or the derivative thereof.
38. The process of paragraph 36 or 37, further comprising the step(s) of:
(c) dissolving or suspending the enantiomerically enriched (S)-pregabalin (L)-tartrate or the derivative thereof in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.
39. The process of paragraph 29, 30 or 34, wherein the acid resolving agent is tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof.
40. The process of paragraph 39, wherein the acid resolving agent is (L)-tartaric acid, O,O′-di-p-toluoyl-(L)-tartaric acid, O,O′-dibenzoyl-(L)-tartaric acid, (S)-3-chloro-mandelic acid, or (S)-3-bromo-mandelic acid.
41. The process of paragraph 40, wherein the acid resolving agent is (L)-tartaric acid.
42. The process of paragraph 30, 32, 35 or 38, wherein the base used in step (c) is an organic and inorganic base.
43. The process of paragraph 42, wherein the organic base is an amine.
44. The process of paragraph 43, wherein the amine is methylamine, dimethylamine, trimethylamine, ethylamine, diethylamine, triethylamine, N,N-diisopropylethylamine, cyclohexylamine, or a mixture thereof.
45. The process of paragraph 42, wherein the inorganic base is ammonia, a metal hydroxide, a metal carbonate, or a mixture thereof.
46. The process of paragraph 45, wherein the metal hydroxide is sodium hydroxide, potassium hydroxide or lithium hydroxide, and/or wherein the metal carbonate is sodium carbonate, lithium carbonate or calcium carbonate.
47. The process of paragraph 30, 32, 35 or 38, wherein in step (d) the enantiomerically enriched γ-amino acid is recrystallised from an organic solvent or water or a mixture thereof.
48. The process of paragraph 30, 32, 35, 38 or 47, wherein the organic solvent used in step (c) or (d) is a protic or aprotic solvent.
49. The process of paragraph 30, 32, 35, 38 or 47, wherein the organic solvent used in step (c) or (d) is an alcohol, a ketone, an ether, an alkane, a cycloalkane, a formamide, an acetate, or a halogenated solvent.
50. A process of preparing an enantiomerically enriched γ-amino acid, comprising the step of:
(c) dissolving or suspending an enantiomerically enriched γ-amino acid salt in an organic solvent or water or a mixture thereof and adjusting the pH of the solution or suspension with a base to obtain enantiomerically enriched γ-amino acid.
51. The process of paragraph 50, wherein the enantiomerically enriched γ-amino acid salt is an (S)-pregabalin salt, and wherein the enantiomerically enriched γ-amino acid is (S)-pregabalin.
52. The process of paragraph 50 or 51, wherein the enantiomerically enriched γ-amino acid salt is a salt of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof.
53. The process of paragraph 52, wherein the enantiomerically enriched γ-amino acid salt is a salt of (L)-tartaric acid, O,O′-di-p-toluoyl-(L)-tartaric acid, O,O′-dibenzoyl-(L)-tartaric acid, (S)-3-chloro-mandelic acid, or (S)-3-bromo-mandelic acid.
54. The process of paragraph 53, wherein the enantiomerically enriched γ-amino acid salt is a salt of (L)-tartaric acid.
55. The process of any one of paragraphs 50 to 54, wherein step (c) is preceded by the step(s) of:
(a) treating a racemic γ-amino acid with an enantiomer of an acid resolving agent to obtain an enantiomerically enriched γ-amino acid salt; and
(b) optionally recrystallising the enantiomerically enriched γ-amino acid salt.
56. The process of any one of paragraphs 50 to 54, wherein step (c) is preceded by the steps of:
(a) treating a racemic γ-amino acid with an enantiomer of an acid resolving agent to obtain a 7-amino acid salt, wherein the γ-amino acid in the γ-amino acid salt is racemic; and
(b) recrystallising the γ-amino acid salt to obtain enantiomerically enriched γ-amino acid salt.
57. The process of paragraph 55 or 56, wherein the acid resolving agent is tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof.
58. The process of paragraph 57, wherein the acid resolving agent is (L)-tartaric acid, O,O′-di-p-toluoyl-(L)-tartaric acid, O,O′-dibenzoyl-(L)-tartaric acid, (S)-3-chloro-mandelic acid, or (S)-3-bromo-mandelic acid.
59. The process of paragraph 58, wherein the acid resolving agent is (L)-tartaric acid.
60. The process of any one of paragraphs 50 to 59, further comprising the step of:
(d) recrystallising the enantiomerically enriched γ-amino acid.
61. The process of any one of paragraphs 50 to 60, comprising the step of:
(c) dissolving or suspending enantiomerically enriched (S)-pregabalin (L)-tartrate or a derivative thereof in an organic solvent or water or a mixture thereof and adjusting the pH of the solution or suspension with a base to obtain enantiomerically enriched (S)-pregabalin.
62. The process of paragraph 61, wherein step (c) is preceded by the step(s) of:
(a) treating racemic pregabalin with (L)-tartaric acid or a derivative thereof to obtain enantiomerically enriched (S)-pregabalin (L)-tartrate or a derivative thereof; and
(b) optionally recrystallising the enantiomerically enriched (S)-pregabalin (L)-tartrate or the derivative thereof.
63. The process of paragraph 61, wherein step (c) is preceded by the steps of:
(a) treating racemic pregabalin with (L)-tartaric acid or a derivative thereof to obtain pregabalin (L)-tartrate or a derivative thereof, wherein the pregabalin in the pregabalin (L)-tartrate or the derivative thereof is racemic; and
(b) recrystallising the pregabalin (L)-tartrate or the derivative thereof to obtain enantiomerically enriched (S)-pregabalin (L)-tartrate or a derivative thereof.
64. The process of any one of paragraphs 61 to 63, further comprising the step of:
(d) recrystallising the enantiomerically enriched (S)-pregabalin.
65. The process of any one of paragraphs 50 to 64, wherein the base used in step (c) is an organic and inorganic base.
66. The process of paragraph 65, wherein the organic base is an amine.
67. The process of paragraph 66, wherein the amine is methylamine, dimethylamine, trimethylamine, ethylamine, diethylamine, triethylamine, N,N-diisopropylethylamine, cyclohexylamine, or a mixture thereof.
68. The process of paragraph 65, wherein the inorganic base is ammonia, a metal hydroxide, a metal carbonate, or a mixture thereof.
69. The process of paragraph 68, wherein the metal hydroxide is sodium hydroxide, potassium hydroxide or lithium hydroxide, and/or wherein the metal carbonate is sodium carbonate, lithium carbonate or calcium carbonate.
70. The process of paragraph 55, 56, 62 or 63, wherein step (a) is carried out in an organic solvent in the presence or absence of water.
71. The process of paragraph 55, 56, 62 or 63, wherein in step (b) the enantiomerically enriched γ-amino acid salt is recrystallised from an organic solvent in the presence or absence of water.
72. The process of paragraph 60 or 64, wherein in step (d) the enantiomerically enriched γ-amino acid is recrystallised from an organic solvent or water or a mixture thereof.
73. The process of any one of paragraphs 50 to 72, wherein the organic solvent is a protic or aprotic solvent.
74. The process of any one of paragraphs 50 to 73, wherein the organic solvent is an alcohol, a ketone, an ether, an alkane, a cycloalkane, a formamide, an acetate, or a halogenated solvent.
75. The process of any one of the preceding paragraphs, wherein the enantiomerically enriched γ-amino acid is prepared on an industrial scale.
76. The process of any one of the preceding paragraphs, wherein the reaction temperature throughout the reaction is less than 80° C.
77. The process of any one of the preceding paragraphs, wherein the total reaction time is less than 30 hours.
78. The process of any one of the preceding paragraphs, wherein the enantiomerically enriched γ-amino acid salt is obtained in a molar yield of 80% or more.
79. The process of any one of the preceding paragraphs, wherein the enantiomerically enriched γ-amino acid salt obtained has an enantiomeric purity of 95% or more (as measured by chiral HPLC).
80. The process of any one of the preceding paragraphs, wherein the enantiomerically enriched γ-amino acid salt obtained has a chemical purity of 95% or more (as measured by HPLC).
81. The process of any one of the preceding paragraphs, wherein the enantiomerically enriched γ-amino acid is obtained in a molar yield of 80% or more.
82. The process of any one of the preceding paragraphs, wherein the enantiomerically enriched γ-amino acid obtained has an enantiomeric purity of 99% or more (as measured by chiral HPLC).
83. The process of any one of the preceding paragraphs, wherein the enantiomerically enriched γ-amino acid obtained has a chemical purity of 99% or more (as measured by HPLC).
84. An enantiomerically enriched γ-amino acid obtained by the process as paragraphed in any one of the preceding paragraphs.
85. A γ-amino acid, having an enantiomeric purity of 99% or more (as measured by chiral HPLC).
86. A γ-amino acid, having a chemical purity of 99% or more (as measured by HPLC).
87. A γ-amino acid salt, having an enantiomeric purity of 95% or more (as measured by chiral HPLC).
88. A γ-amino acid salt, having a chemical purity of 95% or more (as measured by HPLC).
89. A pharmaceutical composition, comprising the γ-amino acid as paragraphed in any one of paragraphs 84 to 86, or the γ-amino acid salt as paragraphed in paragraph 87 or 88.

### EXAMPLES

### Example 1

### Resolution of Racemic Pregabalin (2) by the Method Depicted in Scheme 2 Using L-Tartaric Acid

### Preparation of (S)-pregabalin (L)-tartrate (4):

A mixture of racemic 3-aminoethyl-5-methylhexanoic acid (2) (100 g), L-tartaric acid (94.3 g), n-butanol (1.0 l) and water (200 ml) was stirred at 25-30° C. to obtain a clear solution. Then the clear solution was filtered, cooled to 8-10° C., and stirred at 8-10° C. for 4 hours. The solid obtained was filtered off and dried under vacuum at 40-45° C. Yield: 90 g (93% molar, 46% w/w). Enantiomeric purity: 99.0% S-isomer (as measured by chiral HPLC). Chemical purity: 99.64% (as measured by HPLC). No lactam impurity was observed by HPLC.

### Recrystallisation of (S)-pregabalin (L)-tartrate (4):

(S)-pregabalin (L)-tartrate (4) (90 g) was charged in n-butanol (450 ml) and water (90 ml) and stirred at 20-30° C. to obtain a clear solution. The clear solution was filtered, cooled to 10-15° C., and stirred for 4 hours. The solid obtained was filtered off and dried under vacuum at 40-45° C. Yield: 81 g (90% molar and w/w). Enantiomeric purity: 100% S-isomer (as measured by chiral HPLC). Chemical purity: 99.84% (as measured by HPLC). No lactam impurity was observed by HPLC.

### Preparation of (S)-pregabalin (1):

Recrystallised (S)-pregabalin (L)-tartrate (4) (81 g) was charged in isopropanol (324 ml) and water (81 ml) and stirred at 25-30° C. to obtain a clear solution. The solution was cooled to 20-25° C. and the pH was adjusted to 7.0 to 7.5 using N,N-diisopropylethylamine (89.1 ml). A solid precipitated out. The reaction mixture was cooled to 0-5° C. and stirred for 1 hour. Then the solid obtained was filtered off and dried under vacuum at 40-45° C. Yield: 36.5 g (88% molar and w/w). Enantiomeric purity: 100% S-isomer (as measured by chiral HPLC). Chemical purity: 99.96% (as measured by HPLC). No lactam impurity was observed by HPLC.

### Recrystallisation of (S)-pregabalin (1):

(S)-Pregabalin (1) (36.5 g) was charged in isopropanol (694 ml) and water (219 ml) and warmed to 30-35° C. to obtain a clear solution. The clear solution was filtered, cooled to 0-5° C., and stirred at 0-5° C. for 4 hours. The solid obtained was filtered off and dried under vacuum at 40-45° C. Yield: 32.8 g (90% molar and w/w). Enantiomeric purity: 100% S-isomer (as measured by chiral HPLC). Chemical purity: 99.99% (as measured by HPLC). No lactam impurity was observed by HPLC.

### Example 2

### Resolution of Racemic Pregabalin (2) by the Method Depicted in Scheme 3 Using L-Tartaric Acid

Preparation of pregabalin (L)-tartrate (5):

A mixture of racemic 3-aminoethyl-5-methylhexanoic acid (2) (100 g), L-tartaric acid (94.3 g), n-butanol (1.0 l) and water (100 ml) was stirred at 20-25° C. to obtain a clear solution. Then the clear solution was filtered, cooled to 0-5° C., and stirred at 0-5° C. for 4 hours. The solid obtained was filtered off and dried under vacuum at 40-45° C. Yield: 184.5 g (95% molar and w/w). Enantiomeric purity: 48% (S)-pregabalin (L)-tartrate and 52% (R)-pregabalin (L)-tartrate (as measured by chiral HPLC). Chemical purity: 99.91% (as measured by HPLC). No lactam impurity was observed by HPLC.

### Preparation of (S)-pregabalin (L)-tartrate (4) by Fractional Crystallisation:

Pregabalin (L)-tartrate (5) (184.5 g) was charged in n-butanol (1845 ml) and water (369 ml) and stirred at 20-30° C. to obtain a clear solution. The clear solution was filtered, cooled to 10-15° C., and stirred for 4 hours. The solid obtained was filtered off and dried under vacuum at 40-45° C. Yield: 81 g (88% molar, 44% w/w). Enantiomeric purity: 99.5% S-isomer (as measured by chiral HPLC). Chemical purity: 99.97% (as measured by HPLC). No lactam impurity was observed by HPLC.

### Preparation of (S)-pregabalin (1):

(S)-pregabalin (L)-tartrate (4) (81 g) was charged in isopropanol (324 ml) and water (81 ml) and stirred at 25-30° C. to obtain a clear solution. The solution was cooled to 20-25° C. and the pH was adjusted to 7.0 to 7.5 using N,N-diisopropylethylamine (89.1 ml). A solid precipitated out. The reaction mixture was cooled to 0-5° C. and stirred for 1 hour. Then the solid obtained was filtered off and dried under vacuum at 40-45° C. Yield: 36.5 g (88% molar and w/w). Enantiomeric purity: 100% S-isomer (as measured by chiral HPLC). Chemical purity: 99.92% (as measured by HPLC). No lactam impurity was observed by HPLC.

### Recrystallisation of S-pregabalin (1):

(S)-Pregabalin (1) (36.5 g) was charged in isopropanol (694 ml) and water (219 ml) and warmed to 30-35° C. to obtain a clear solution. The clear solution was filtered, cooled to 0-5° C., and stirred at 0-5° C. for 4 hours. The solid obtained was filtered off and dried under vacuum at 40-45° C. Yield: 32.8 g (90% molar and w/w). Enantiomeric purity: 100% S-isomer (as measured by chiral HPLC). Chemical purity: 99.98% (as measured by HPLC). No lactam impurity was observed by HPLC.

### Example 3

### Resolution of Racemic Pregabalin (2) by a Method Similar to the One Depicted in Scheme 2 but Using O,O′-di-p-Toluoyl-(D)-Tartaric Acid

Preparation of (S)-pregabalin O,O′-di-p-toluoyl-(D)-tartrate (6):

A mixture of racemic 3-aminoethyl-5-methylhexanoic acid (2) (100 g), O,O′-di-p-toluoyl-(D)-tartaric acid (126 g), t-butanol (1.0 l) and water (200 ml) was stirred at 20-25° C. to obtain a clear solution. Then the clear solution was filtered, cooled to 0-5° C., and stirred at 0-5° C. for 4 hours. The solid obtained was filtered off and dried under vacuum at 40-45° C. Yield: 168 g (98% molar, 49% w/w). Enantiomeric purity: 96.0% S-isomer (as measured by chiral HPLC). Chemical purity: 99.50% (as measured by HPLC). No lactam impurity was observed by HPLC.

Recrystallisation of (S)-pregabalin O,O′-di-p-toluoyl-(D)-tartrate (6):

(S)-pregabalin O,O′-di-p-toluoyl-(D)-tartrate (6) (168 g) was charged in t-butanol (1680 ml) and water (336 ml) and stirred at 25-30° C. to obtain a clear solution. The clear solution was filtered, cooled to 10-15° C., and stirred for 4 hours. The solid obtained was filtered off and dried under vacuum at 40-45° C. Yield: 148 g (88% molar and w/w). Enantiomeric purity: 99.8% S-isomer (as measured by chiral HPLC). Chemical purity: 99.50% (as measured by HPLC). No lactam impurity was observed by HPLC.

### Preparation of (S)-pregabalin (1):

Recrystallised (S)-pregabalin O,O′-di-p-toluoyl-(D)-tartrate (6) (148 g) was charged in isopropanol (740 ml) and water (196 ml) and stirred at 25-30° C. to obtain a clear solution. The solution was cooled to 20-25° C. and the pH was adjusted to 7.0 to 7.5 by using N,N-diisopropylethylamine (70.0 ml). A solid precipitated out. The reaction mixture was cooled to 0-5° C. and stirred for 1 hour. Then the solid obtained was filtered off and dried under vacuum at 40-45° C. Yield: 37.5 g (87% molar and w/w). Enantiomeric purity: 100% S-isomer (as measured by chiral HPLC). Chemical purity: 99.96% (as measured by HPLC). No lactam impurity was observed by HPLC.

### Recrystallisation of (S)-pregabalin (1):

(S)-Pregabalin (1) (37.5 g) was charged in isopropanol (694 ml) and water (219 ml) and warmed to 30-35° C. to obtain a clear solution. The clear solution was filtered, cooled to 0-5° C., and stirred at 0-5° C. for 4 hours. The solid obtained was filtered off and dried under vacuum at 40-45° C. Yield: 32.8 g (87.5% molar and w/w). Enantiomeric purity: 100% S-isomer (as measured by chiral HPLC). Chemical purity: 99.98% (as measured by HPLC). No lactam impurity was observed by HPLC.

Steps 1 and 2 of example 3 were also carried out using n-butanol and similar results were obtained.

### Example 4

### Resolution of Racemic Pregabalin (2) by a Method Similar to the One Depicted in Scheme 2 Using L-Tartaric Acid

Example 1 was repeated, but using the solvents indicated in Table 1 in the resolution step (1) and the fractional crystallisation step (2). The enantiomeric and chemical purities of the (S)-pregabalin (1) obtained using these solvents are also indicated in Table 1.

TABLE 1

| | Enantiomeric purity | |
|---|---|---|
| | (as measured by chiral | Chemical purity |
| Solvent | HPLC) | (as measured by HPLC) |
| ethanol | >99.5% | >99.90% |
| isopropanol | >99.5% | >99.90% |
| t-butanol:ethanol (3:1) | >99.8% | >99.95% |

It will be understood that the present invention has been described above by way of example only. The examples are not intended to limit the scope of the invention. Various modifications and embodiments can be made without departing from the scope and spirit of the invention, which is defined by the following claims only.

1-89. (canceled)

90. A process of preparing an enantiomerically enriched γ-amino acid, comprising the step of treating a racemic γ-amino acid with an enantiomer of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, or a derivative thereof, or an enantiomer of a mandelic acid derivative, to provide an enantiomerically enriched γ-amino acid.

91. The process of claim 90, wherein:
(i) the enantiomerically enriched γ-amino acid is enantiomerically enriched (S)-pregabalin; and/or
(ii) the enantiomer of tartaric acid or a derivative thereof is (L)-tartaric acid, O,O′-di-p-toluoyl-(L)-tartaric acid or O,O′-dibenzoyl-(L)-tartaric acid; and/or
(iii) the enantiomer of tartaric acid or a derivative thereof is (L)-tartaric acid; and/or
(iv) the enantiomer of a mandelic acid derivative is (S)-3-chloro-mandelic acid or (S)-3-bromo-mandelic acid.

92. The process of claim 90, comprising the steps of:
(a) treating a racemic γ-amino acid with an enantiomer of tartaric acid or a derivative thereof to obtain an enantiomerically enriched γ-amino acid salt;
(b) optionally recrystallising the enantiomerically enriched γ-amino acid salt;
(c) dissolving or suspending the enantiomerically enriched γ-amino acid salt obtained in step (a) or (b) in an organic solvent or water or a mixture thereof and adjusting the pH of the solution or suspension with a base to obtain enantiomerically enriched γ-amino acid; and
(d) optionally recrystallising the enantiomerically enriched γ-amino acid.

93. The process of claim 93, comprising the steps of:
(a) treating racemic pregabalin with (L)-tartaric acid or a derivative thereof to obtain enantiomerically enriched (S)-pregabalin (L)-tartrate or a derivative thereof;
(b) optionally recrystallising the enantiomerically enriched (S)-pregabalin (L)-tartrate or the derivative thereof;
(c) dissolving or suspending the enantiomerically enriched (S)-pregabalin (L)-tartrate or the derivative thereof obtained in step (a) or (b) in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

94. The process of claim 90, comprising the steps of:
(a) treating a racemic γ-amino acid with an enantiomer of tartaric acid or a derivative thereof to obtain a γ-amino acid salt, wherein the γ-amino acid in the γ-amino acid salt is racemic;
(b) recrystallising the γ-amino acid salt to obtain enantiomerically enriched γ-amino acid salt;
(c) dissolving or suspending the enantiomerically enriched γ-amino acid salt in an organic solvent or water or a mixture thereof and adjusting the pH of the solution or suspension with a base to obtain enantiomerically enriched γ-amino acid; and
(d) optionally recrystallising the enantiomerically enriched γ-amino acid.

95. The process of claim 95, comprising the steps of:
(a) treating racemic pregabalin with (L)-tartaric acid or a derivative thereof to obtain pregabalin (L)-tartrate or a derivative thereof, wherein the pregabalin in the pregabalin (L)-tartrate or the derivative thereof is racemic;
(b) recrystallising the pregabalin (L)-tartrate or the derivative thereof to obtain enantiomerically enriched (S)-pregabalin (L)-tartrate or a derivative thereof;
(c) dissolving or suspending the enantiomerically enriched (S)-pregabalin (L)-tartrate or the derivative thereof in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

96. A process of preparing an enantiomerically enriched γ-amino acid salt, comprising crystallising the enantiomerically enriched γ-amino acid salt from a solvent mixture comprising an alcohol and at least 15% water.

97. The process of claim 97, wherein:
(i) the solvent mixture comprises alcohol:water in a ratio of 85:15 to 50:50; and/or
(ii) the solvent mixture comprises alcohol:water in a ratio of 80:20 to 60:40; and/or
(iii) the alcohol is methanol, ethanol, n-propanol, i-propanol, n-butanol, i-butanol, t-butanol, 2-pentanol, 3-pentanol, 4-penten-2-ol, 1,6-hexanediol, 1-hexanol, 5-hexen-1-ol, glycerol, 1-heptanol, 2-heptanol, 1-octanol, 2-octanol, or 3-octanol; and/or
(iv) the alcohol is n-butanol.

98. The process of claim 97, wherein the enantiomerically enriched γ-amino acid salt is:
an (S)-pregabalin salt; and/or
(ii) a salt of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof; and/or
(iii) a salt of (L)-tartaric acid, O,O′-di-p-toluoyl-(L)-tartaric acid, O,O′-dibenzoyl-(L)-tartaric acid, (S)-3-chloro-mandelic acid, or (S)-3-bromo-mandelic acid; and/or
(iv) a salt of (L)-tartaric acid.

99. The process of claim 97, comprising the steps of:
(a) treating a racemic γ-amino acid with an enantiomer of an acid resolving agent in a solvent mixture comprising an alcohol and at least 15% water to obtain an enantiomerically enriched γ-amino acid salt, wherein the enantiomerically enriched γ-amino acid salt crystallises from the solvent mixture;
(b) optionally recrystallising the enantiomerically enriched γ-amino acid salt from a solvent mixture comprising an alcohol and at least 15% water;
(c) dissolving or suspending the enantiomerically enriched γ-amino acid salt obtained in step (a) or (b) in an organic solvent or water or a mixture thereof and adjusting the pH of the solution or suspension with a base to obtain enantiomerically enriched γ-amino acid; and
(d) optionally recrystallising the enantiomerically enriched γ-amino acid.

100. The process of claim 100, comprising the step of:
(a) treating racemic pregabalin with (L)-tartaric acid or a derivative thereof in a solvent mixture comprising an alcohol and at least 15% water to obtain enantiomerically enriched (S)-pregabalin (L)-tartrate or a derivative thereof, wherein the enantiomerically enriched (S)-pregabalin (L)-tartrate or the derivative thereof crystallises from the solvent mixture;
(b) optionally recrystallising the enantiomerically enriched (S)-pregabalin (L)-tartrate or the derivative thereof from a solvent mixture comprising an alcohol and at least 15% water;
(c) dissolving or suspending the enantiomerically enriched (S)-pregabalin (L)-tartrate or the derivative thereof obtained in step (a) or (b) in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

101. The process of claim 97, comprising the steps of:
(a) treating a racemic γ-amino acid with an enantiomer of an acid resolving agent to obtain a γ-amino acid salt, wherein the γ-amino acid in the γ-amino acid salt is racemic;
(b) recrystallising the γ-amino acid salt from a solvent mixture comprising an alcohol and at least 15% water to obtain enantiomerically enriched γ-amino acid salt;
(c) dissolving or suspending the enantiomerically enriched γ-amino acid salt in an organic solvent or water or a mixture thereof and adjusting the pH of the solution or suspension with a base to obtain enantiomerically enriched γ-amino acid; and
(d) optionally recrystallising the enantiomerically enriched γ-amino acid.

102. The process of claim 101, comprising the step of:
(a) treating racemic pregabalin with (L)-tartaric acid or a derivative thereof to obtain pregabalin (L)-tartrate or a derivative thereof, wherein the pregabalin in the pregabalin (L)-tartrate or the derivative thereof is racemic;
(b) recrystallising the pregabalin (L)-tartrate or the derivative thereof from a solvent mixture comprising an alcohol and at least 15% water to obtain enantiomerically enriched (s)-pregabalin (L)-tartrate or a derivative thereof;
(c) dissolving or suspending the enantiomerically enriched (S)-pregabalin (L)-tartrate or the derivative thereof in an organic solvent or water or a mixture thereof and adjusting the pH of the solution or suspension with a base to obtain enantiomerically enriched (S)-pregabalin; and
(d) optionally recrystallising the enantiomerically enriched (S)-pregabalin.

103. A process of preparing an enantiomerically enriched γ-amino acid, comprising the step of:
(c) dissolving or suspending an enantiomerically enriched γ-amino acid salt in an organic solvent or water or a mixture thereof and adjusting the pH of the solution or suspension with a base to obtain enantiomerically enriched γ-amino acid.

104. The process of claim 103, wherein the enantiomerically enriched γ-amino acid salt is:
(i) an (S)-pregabalin salt, and wherein the enantiomerically enriched γ-amino acid is (S)-pregabalin; and/or
(ii) a salt of tartaric acid, camphor-10-sulphonic acid, camphor-3-sulphonic acid, 3-bromo-camphor-9-sulphonic acid, 2-keto-gulonic acid, α-methoxyphenylacetic acid, 2-nitrotartranilic acid, malic acid, 2-phenoxypropionic acid, N-acetylleucine, N-(α-methylbenzyl)succinamic acid, N-(α-methylbenzyl)phthalamic acid, quinic acid, di-O-isopropylidene-2-oxo-L-gulonic acid, 2-hydroxy-4-isopropenyl-1-methyl-cyclohexane-1-sulphonic acid, mandelic acid, or a derivative thereof; and/or
(iii) a salt of (L)-tartaric acid, O,O′-di-p-toluoyl-(L)-tartaric acid, O,O′-dibenzoyl-(L)-tartaric acid, (S)-3-chloro-mandelic acid, or (S)-3-bromo-mandelic acid; and/or
(iv) a salt of (L)-tartaric acid.

105. The process of claim 103, wherein step (c) is preceded by the step(s) of:
(a) treating a racemic γ-amino acid with an enantiomer of an acid resolving agent to obtain an enantiomerically enriched γ-amino acid salt; and
(b) optionally recrystallising the enantiomerically enriched γ-amino acid salt.

106. The process of claim 103, wherein step (c) is preceded by the steps of:
(a) treating a racemic γ-amino acid with an enantiomer of an acid resolving agent to obtain a γ-amino acid salt, wherein the γ-amino acid in the γ-amino acid salt is racemic; and
(b) recrystallising the γ-amino acid salt to obtain enantiomerically enriched γ-amino acid salt.

107. The process of claim 103, further comprising the step of:
(d) recrystallising the enantiomerically enriched γ-amino acid.

108. The process of claim 103, wherein the base used in step (c) is an organic and inorganic base.

109. The process of claim 108, wherein:
(i) the organic base is an amine; and/or
(ii) the organic base is methylamine, dimethylamine, trimethylamine, ethylamine, diethylamine, triethylamine, N,N-diisopropylethylamine, cyclohexylamine, or a mixture thereof; and/or
(iii) the inorganic base is ammonia, a metal hydroxide, a metal carbonate, or a mixture thereof; and/or
(iv) the inorganic base is ammonia, a metal hydroxide, a metal carbonate, or a mixture thereof, and wherein the metal hydroxide is sodium hydroxide, potassium hydroxide or lithium hydroxide, and/or wherein the metal carbonate is sodium carbonate, lithium carbonate or calcium carbonate.

110. The process of claim 103, wherein:
(i) the organic solvent is a protic or aprotic solvent; and/or
(ii) the organic solvent is an alcohol, a ketone, an ether, an alkane, a cycloalkane, a formamide, an acetate, or a halogenated solvent.

111. The process of claim 103, comprising the step of:
(c) dissolving or suspending enantiomerically enriched (S)-pregabalin (L)-tartrate or a derivative thereof in an organic solvent or water or a mixture thereof and adjusting the pH of the solution or suspension with a base to obtain enantiomerically enriched (S)-pregabalin.

112. An enantiomerically enriched γ-amino acid obtained by the process as claimed in claim 90.

113. An enantiomerically enriched γ-amino acid obtained by the process as claimed in claim 97.

114. An enantiomerically enriched γ-amino acid obtained by the process as claimed in claim 103.

115. A γ-amino acid, having an enantiomeric purity of 99% or more (as measured by chiral HPLC).

116. A γ-amino acid, having a chemical purity of 99% or more (as measured by HPLC).

117. A γ-amino acid salt, having an enantiomeric purity of 95% or more (as measured by chiral HPLC).

118. A γ-amino acid salt, having a chemical purity of 95% or more (as measured by HPLC).

119. A pharmaceutical composition, comprising the γ-amino acid as claimed in claim 113.

120. A pharmaceutical composition, comprising the γ-amino acid as claimed in claim 114.

121. A pharmaceutical composition, comprising the γ-amino acid as claimed in claim 115.

122. A pharmaceutical composition, comprising the γ-amino acid as claimed in claim 116.

123. A pharmaceutical composition, comprising the γ-amino acid as claimed in claim 117.

124. A pharmaceutical composition, comprising the γ-amino acid salt as claimed in claim 118.

125. A pharmaceutical composition, comprising the γ-amino acid salt as claimed in claim 119.
